(19) United States
(12) Patent Application Publication
(10) Pub. No.: US 2018/0326055 A1
(43) Pub. Date: Nov. 15, 2018

(54) NANOSTRUCTURE

(71) Applicant: RIKEN, Saitama (JP)

(72) Inventors: Motoki UEDA, Saitama (JP); Yoshihiro ITO, Saitama (JP)

(21) Appl. No.: 15/977,289

(22) Filed: May 11, 2018

(30) Foreign Application Priority Data
May 12, 2017 (JP) 2017-096075

(51) Int. Cl. A61K 39/395; A61K 47/54; A61K 47/69; A61K 9/127

(52) U.S. Cl. CPC A61K 39/3955; A61K 9/1271; A61K 47/6911; A61K 47/544

(57) ABSTRACT

In order to obtain a nanostructure that encapsulates an agent therein and that can be easily taken up by cells, a nanostructure of the present invention is a hollow body constituted by a wall formed from an assembly of amphiphilic molecules containing a hydrophilic block and a hydrophobic block, the hollow body having an aspect ratio greater than 1.0, the nanostructure encapsulating an agent therein.

### TECHNICAL FIELD

The present invention relates to a nanostructure encapsulating an agent therein.

### BACKGROUND ART

As nanotechnology has become increasingly important in recent years, various new functional materials, which make use of the properties specific to nano-sized substances, have been developed. Such nano-sized functional materials have been promising in applications to various fields such as energy, electronics, and pharmaceuticals. For example, in the field of pharmaceuticals, liposome, which is a nanoparticle composed of phospholipid, or the like is used as a carrier in drug delivery system (DDS).

Furthermore, in regard to a nanostructure formed from peptides, Non-patent Literature 1 states that peptide nanostructures of various shapes were prepared from amphiphilic peptide chains having a hydrophilic block and a hydrophobic helical block.

### CITATION LIST

### Non-Patent Literature

[Non-patent Literature 1]
M Ueda et al., Polymer Journal, 45, 509-515 (2013)

### SUMMARY OF INVENTION

### Technical Problem

There is a demand for development of a carrier for efficient delivery of a drug into cells. Under such circumstances, it is an object of one aspect of the present invention to obtain a nanostructure that encapsulates an agent therein and that can be easily taken up by cells.

It should be noted that Non-patent Literature 1 merely teaches that nanostructures of various shapes were prepared, and does not mention any specific usefulness in any field.

### Solution to Problem

In order to attain the above object, one aspect of the present invention includes the following.

(1) A nanostructure which is a hollow body constituted by a wall formed from an assembly of amphiphilic molecules containing a hydrophilic block and a hydrophobic block, the hollow body having an aspect ratio greater than 1.0,

the nanostructure encapsulating an agent therein.

### Advantageous Effects of Invention

According to one aspect of the present invention, it is possible to obtain a nanostructure that encapsulates an agent therein and that can be easily taken up by cells.

### BRIEF DESCRIPTION OF DRAWINGS

FIG. 1 schematically illustrates examples of a method of producing a nanostructure.

FIG. 2 shows TEM images taken in Example 1.

FIG. 3 schematically illustrates a test method in Example 2 and shows the results of Example 2.

FIG. 4 shows the results of Reference Example 1.

FIG. 5 shows the results of Reference Example 2.

FIG. 6 shows the results of Reference Example 3.

FIG. 7 shows the results of Example 3.

FIG. 8 shows the results of Example 4.

FIG. 9 shows the results of Example 5.

FIG. 10 shows the results of Example 6.

FIG. 11 shows the results of Example 7.

### DESCRIPTION OF EMBODIMENTS

(Outline)

A nanostructure in accordance with one embodiment of the present invention is a hollow body constituted by a wall formed from an assembly of amphiphilic molecules containing a hydrophilic block and a hydrophobic block, the hollow body having an aspect ratio greater than 1.0, and the nanostructure encapsulates an agent therein.

(Amphiphilic Molecule)

Examples of amphiphilic molecules include amphiphilic peptide chains and lipids.

The term “hydrophilic block” refers to a region that shows hydrophilicity. There is no particular limitation on the degree of the physical property “hydrophilic” of the hydrophilic block. The hydrophilic block needs only be hydrophilic to the extent that the hydrophilic block is more hydrophilic than other regions of an amphiphilic molecule and that the amphiphilic molecule, constituted by the hydrophilic block and the other regions, as a whole can be amphiphilic. Alternatively, the hydrophilic block needs only be hydrophilic to the extent that the amphiphilic molecules are capable of becoming self-organized and forming a self-assembly in a medium.

The term “hydrophobic block” refers to a region that shows hydrophobicity. There is no particular limitation on the degree of the physical property “hydrophobic” of the hydrophobic block. The hydrophobic block needs only be hydrophobic to the extent that the hydrophobic block is more hydrophobic than other regions of an amphiphilic molecule and that the amphiphilic molecule, constituted by the hydrophobic block and the other regions, as a whole can be hydrophobic. Alternatively, the hydrophobic block needs only be hydrophobic to the extent that the amphiphilic molecules are capable of becoming self-organized and forming a self-assembly in a medium.

(Amphiphilic Peptide Chain)

In this specification, the term “peptide” refers to a compound formed from two or more amino acids bound together by a peptide bond. In this specification, the term “amino acid” is a concept that includes natural amino acids, unnatural amino acids, and derivatives thereof resulting from modification and/or chemical change. The concept also includes α-amino acids, β-amino acids, γ-amino acids, and the like. The amino acid is preferably an α-amino acid. In the present invention, the term “amphiphilic peptide chain” is a peptide-based amphiphilic molecule, which may partially contain a constituent other than peptide. Examples of such a constituent include modification at N-terminus or C-terminus and non-peptide linker between blocks.

The term “hydrophilic peptide block” refers to a region that shows hydrophilicity, and may partially contain a constituent other than peptide. There is no particular limitation on the degree of the physical property “hydrophilic” of the hydrophilic peptide block. The hydrophilic peptide block needs only be hydrophilic to the extent that the hydrophilic peptide block is more hydrophilic than other regions of an amphiphilic peptide chain and that the amphiphilic peptide chain, constituted by the hydrophilic peptide block and the other regions, as a whole can be amphiphilic. Alternatively, the hydrophilic peptide block needs only be hydrophilic to the extent that the amphiphilic peptide chains are capable of becoming self-organized and forming a self-assembly in a medium.

The amino acids constituting the hydrophilic peptide block are not limited to a particular kind. Examples of the amino acids constituting the hydrophilic peptide block include N-methylglycine (sarcosine), lysine, and histidine. The “hydrophilicity” may be achieved by, for example, hydrogen bonds formed by side chains of the amino acids constituting the hydrophilic peptide block, or may be achieved by hydrogen bonds formed by carbonyl of the main chains of the amino acids constituting the hydrophilic peptide block. The amino acids constituting the hydrophilic peptide block are preferably nonionic (uncharged) amino acids. The hydrophilicity obtained by hydration is advantageous in that this makes it easy to control the shape of a self-assembly by selecting the length of the hydrophilic peptide block, because the hydrophilicity obtained by hydration is weaker than that obtained by ions. The hydrophilicity obtained by hydration is advantageous also in that the surface of a nanostructure is covered with a nonionic polymer and thereby the nanostructure is not easily recognized as foreign in vivo. A preferred one of such nonionic amino acids is sarcosine.

The hydrophilic peptide block may be constituted by amino acids of two or more kinds. The kinds and proportions of the amino acids constituting the hydrophilic peptide block are selected appropriately by a person skilled in the art so that the hydrophilic peptide block as a whole is hydrophilic.

The number of the amino acids constituting the hydrophilic peptide block is not particularly limited, and is preferably 5 to 80, more preferably 15 to 40, even more preferably 20 to 35, and, in one example, particularly preferably 30. In a case where the number of the amino acids is 5 or more, the hydrophilic peptide block is hydrophilic enough and a self-assembly can easily form a desired shape. In a case where the number of the amino acids is equal to or less than 80, the hydrophilic block does not become too large and a self-assembly can easily form a desired shape.

The term “hydrophobic peptide block” refers to a region that shows hydrophobicity, and may partially contain a constituent other than peptide. There is no particular limitation on the degree of the physical property “hydrophobic” of the hydrophobic peptide block. The hydrophobic peptide block needs only be hydrophobic to the extent that the hydrophobic peptide block is more hydrophobic than other regions of an amphiphilic peptide chain and that the amphiphilic peptide chain, constituted by the hydrophobic peptide block and the other regions, as a whole can be amphiphilic. Alternatively, the hydrophobic peptide block needs only be hydrophobic to the extent that the amphiphilic peptide chains are capable of becoming self-organized and forming a self-assembly in a medium.

The amino acids constituting the hydrophobic peptide block are not limited to a particular kind, and are preferably hydrophobic amino acids. Examples of the amino acids constituting the hydrophobic peptide block include glycine, alanine, valine, leucine, isoleucine, proline, methionine, tyrosine, tryptophan, aminoisobutyric acid, norleucine, α-aminobutyric acid, and cyclohexylalanine. It is preferable that the hydrophobic peptide block has a helix structure. A hydrophobic peptide block having a helix structure is advantageous in that such blocks are strong in structure and are oriented densely and in parallel. Examples of a hydrophobic peptide block having such a helix structure include poly(leucine-aminoisobutyric acid), polyalanine, polyglycine, and polyproline.

The hydrophobic peptide block may be constituted by amino acids of two or more kinds. The kinds and proportions of the amino acids constituting the hydrophobic peptide block are selected appropriately by a person skilled in the art so that the hydrophobic peptide block as a whole is hydrophobic.

The number of the amino acids constituting the hydrophobic peptide block is not particularly limited, and is preferably 8 to 30, more preferably 8 to 20, even more preferably 12 to 16, and, in one example, particularly preferably 12 or 16.

The ratio in length of the hydrophilic peptide block to the hydrophobic peptide block is not particularly limited. It is preferable that the number of amino acids in the hydrophilic peptide block to that of the hydrophobic peptide block is 1:1 to 3:1.

Either of the hydrophilic and hydrophobic peptide blocks can be located on the N-terminus side. In order to achieve easy synthesis, it is preferable that the hydrophilic peptide block is located on the N-terminus side.

The hydrophilic peptide block and the hydrophobic peptide block may be bound together by a linker or may be bound together directly without linkers. The linker may be a linker constituted by peptide or a non-peptide linker.

The N-terminus and C-terminus of the amphiphilic peptide chain are preferably modified (protected) from stability point of view (i.e., in order to obtain a peptide nanostructure whose molecules are not changed by pH, temperature, and/or the like conditions and which is stable to external environment changes). The N-terminus or C-terminus of the amphiphilic peptide chain may be labeled with a fluorescent material or the like which is bound to the terminus.

In one preferred example, the hydrophilic peptide block of the amphiphilic peptide chain contains sarcosine as repeating unit, and the hydrophobic peptide block contains (leucine-aminoisobutyric acid) as repeating unit. In a more preferred example, the amphiphilic peptide chain is preferably represented by the following Formula (I). In Formula (I), m is not particularly limited and is preferably 5 to 80, more preferably 15 to 40, even more preferably 20 to 35 and, in a particularly preferred example, 30. In Formula (I), n is not particularly limited and is preferably 4 to 15, more preferably 4 to 10, even more preferably 6 to 8 and, in a particularly preferred example, 6 or 8. The poly(leucine-aminoisobutyric acid) of the hydrophobic peptide block forms a helix structure in a case where leucines have structures with the same chirality. In one example, each leucine is L-leucine. In Formula (I), R₁ is not particularly limited and is, for example, a non-reactive protecting group, specifically ketole group, acetyl group, or the like. R₂ is not particularly limited and is, for example, a non-reactive protecting group, specifically an alkoxy group (e.g., C1-C4 alkoxy group), benzyl ester group, or the like.

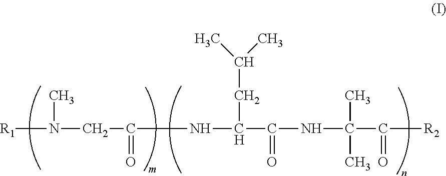

In an even more preferred example, the amphiphilic peptide chain is preferably represented by the following Formula (II), where m and n are as defined in Formula (I).

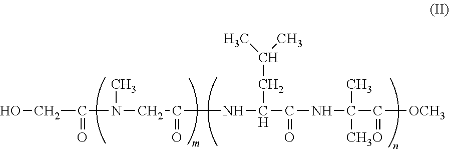

A method of synthesizing an amphiphilic peptide chain is not particularly limited and may be a known peptide synthesis method. The peptide synthesis can be carried out by, for example, peptide condensation using a liquid phase method, or the like.

The amphiphilic peptide chains constituting a peptide nanostructure may be of a single kind or of two or more kinds.

(Hollow Body)

The peptide nanostructure of the present embodiment is a hollow body constituted by a wall formed from an assembly of a plurality of amphiphilic peptide chains. There is no particular limitation on how the amphiphilic peptide chains are assembled and, in one example, the amphiphilic peptide chains are oriented and associated together in a self-assembled manner. In a more specific example, the amphiphilic peptide chains are assembled by hydrophobic interaction. The wall may have a multilayer structure. For example, the following structure can be employed: hydrophilic peptide blocks are arranged to form inner and outer surface layers of the wall; and hydrophobic peptide blocks are arranged to form an internal layer of the wall. More specifically, the wall of the hollow body can be an association of amphiphilic peptide chains arranged such that adjacent amphiphilic peptide chains have their hydrophilic peptide blocks located at opposite ends. As such, the wall can have a three-layer structure consisting of: a first hydrophilic layer constituted by hydrophilic peptide blocks of some amphiphilic peptide chains; a hydrophobic layer constituted by hydrophobic peptide blocks; and a second hydrophilic layer constituted by hydrophilic peptide blocks of the other amphiphilic peptide chains. In such a structure, the outer surface layer of the hollow body is hydrophilic and therefore has good affinity with water. This achieves better adaptability in vivo. Furthermore, since the inner surface layer of the hollow body is hydrophilic, it is possible to suitably encapsulate a hydrophilic agent.

In the present embodiment, the shape of the hollow body is not particularly limited. In order to achieve easy cellular uptake, it is preferable that the hollow body has a tube shape portion (that is, at least part of the wall has a tube shape). In order to better retain the agent, it is preferable that the hollow body has a closed structure.

The tube shape portion can be prepared by, for example, dispersing amphiphilic peptide chains into an aqueous medium to obtain a dispersion and then heating the dispersion. In this arrangement, more specifically, the amphiphilic peptide chains are dispersed in the aqueous medium and thereby form a sheet-shape structure, and then edges of the sheet-shape structure are allowed to associate together, with heat, to form a tube shape. For example, a precursor, which is a helically twisted sheet-shape structure, is formed first, and then edges of the precursor, which have been brought to close to each other by helical twisting, associate together and close to form a tube shape.

In this specification, the term “aqueous medium” refers to a liquid whose main component is water. In this specification, the term “liquid whose main component is water” means that the percentage of the volume of water occupying the liquid is greater than other components, and means that preferably more than 50% but not more than 100% of the total volume of the liquid is water. The aqueous medium is preferably a liquid that is safe for use in vivo, such as physiological saline or distilled water for injection, as well as pH buffer solution or the like.

The amphiphilic peptide chains may be dissolved in an organic solvent (ethanol, dimethylformamide, methanol or the like) to obtain a solution first and then the solution may be added (for example, injected) to the aqueous medium. The organic solvent is preferably a liquid that is safe for use in vivo, and is more preferably ethanol. By dissolving the amphiphilic peptide chains in an organic solvent first, the amphiphilic peptide chains which are dissociated from each other, not in the crystal form, are added into the aqueous medium, and therefore it is possible to allow the amphiphilic peptide chains to form a tube shape portion efficiently. Thus, in one example, the “aqueous medium” can contain such an organic solvent.

In preparation of the tube shape portion, the amount of amphiphilic peptide chain relative to the aqueous medium is not particularly limited, and is preferably 0.1 to 10 mg/mL, more preferably 0.5 to 2 mg/mL, in view of dispersibility in water. The dispersion of the amphiphilic peptide chains into the aqueous medium is carried out preferably at 4 to 25° C. In order to uniformly disperse the amphiphilic peptide chains and to obtain a uniform sheet-shape structure, it is preferable to carry out stirring.

The heating temperature is not particularly limited and is, for example, preferably 30 to 90° C. The heating time is not particularly limited and is, for example, preferably 10 minutes to 24 hours. As will be described later in Examples, the heating temperature and heating time influence the aspect ratio of the resulting tube shape portion. A higher heating temperature tends to provide a higher aspect ratio. A longer heating time tends to provide a higher aspect ratio. In view of this, the heating temperature and heating time may be selected appropriately depending on a desired aspect ratio. In one example, the heating temperature and heating time influence the length of the resulting tube shape portion. When heating temperature is raised and/or heating time is extended, two or more tube shape portions are joined (associated) together at their ends and result in a longer tube shape portion with the same diameter.

An amphiphilic peptide chain suitable for preparation of the tube shape portion is, for example, a compound represented by the foregoing Formula (I) where, in one example, m is preferably 15 to 40, and n is preferably 6 to 8, more preferably 6 or 7.

The tube shape portion is not particularly limited in size and, for example, preferably has an outer diameter of 20 to 200 nm in order to achieve a suitable size for use in vivo. The thickness of the tube shape portion can depend on the length of the amphiphilic peptide chain used, and can be, for example, 5 to 10 nm. The length of the tube shape portion is not particularly limited and is, for example, preferably 10 to 1000 nm, more preferably 20 to 200 nm, in order to achieve easy cellular uptake and accumulation in cancer sites.

The hollow body has an aspect ratio greater than 1.0. Such an aspect ratio enables easy cellular uptake of the nanostructure, as compared to cases in which the nanostructure is in the shape of a sphere (aspect ratio is 1.0). The aspect ratio of the hollow body is preferably 1.2 to 30.0, more preferably 1.5 to 7.0, more preferably 1.5 to 5.0, even more preferably 2.0 to 5.0, particularly preferably 2.4 to 3.8, for achieving easy cellular uptake. As used herein, the “aspect ratio of a hollow body” indicates anisotropy of a structure, and refers to a “dimension along long axis divided by dimension along short axis”. In a case where the hollow body is in a tube shape, the “aspect ratio of a hollow body” refers to “length of tube divided by diameter of cross section of tube”.

There is no particular limitation on structures other than the tube shape portion. In one example, the hollow body can be such that three tube shape portions joined together are extending in three directions. Furthermore, for retaining the encapsulated agent for a longer period of time, the tube shape portion is preferably structured such that at least one end thereof is closed. It is more preferable that the hollow body has a closed structure.

In one example, the nanostructure can be structured such that the ends of the tube shape portion are all closed. The shape of a structure to close an end (such a structure is referred to as “cap portion”) is not particularly limited and, in one example, the cap portion can be in the shape of a partial sphere, preferably a hemisphere. The nanostructure may be in the shape of a dumbbell in which the outer diameter of the cap portion is greater than the outer diameter of the tube shape portion; however, in order to achieve easy cellular uptake and to retain an agent for long time, it is preferable that the peptide nanostructure is in the shape of a capsule constituted by: a tube shape portion; and cap portions in the shape of hemispheres whose diameter (outer diameter) is substantially the same as the outer diameter of the tube shape portion.

In one example, first amphiphilic peptide chains constitute a tube shape portion and second amphiphilic peptide chains constitute a cap portion.

The second amphiphilic peptide chains may be of the same kind as or of a different kind from amphiphilic peptide chains (the first amphiphilic peptide chains) for use in formation of the tube shape portion. The second amphiphilic peptide chains are preferably amphiphilic peptide chains that turn into the shape of a sphere when they are dispersed alone in an aqueous medium and heated in the absence of the tube shape portion.

The second amphiphilic peptide chains may be dissolved in an organic solvent (ethanol, dimethylformamide, methanol or the like) to obtain a solution first and then the solution may be added (for example, injected) to the aqueous medium. The organic solvent is preferably a liquid that is safe for use in vivo, and is more preferably ethanol. By dissolving the second amphiphilic peptide chains in an organic solvent first, the second amphiphilic peptide chains dissociated from each other, not in the crystal form, are added into the aqueous medium, and therefore it is possible to efficiently allow the second amphiphilic peptide chains to form a cap portion. Thus, in one example, the “aqueous medium” can contain such an organic solvent.

The amount of the second amphiphilic peptide chain relative to the amount of the first amphiphilic peptide chain is not particularly limited and is, for example, preferably selected such that the number of ends of tube shape portions and the number of cap portions are the same, in order to efficiently obtain desired structures. In one example, the molar quantity of the second amphiphilic peptide chain is preferably 0.5 to 3 times that of the first amphiphilic peptide chain, more preferably 0.5 to 2 times that of the first amphiphilic peptide chain and, for achieving higher yield, even more preferably 2 times that of the first amphiphilic peptide chain.

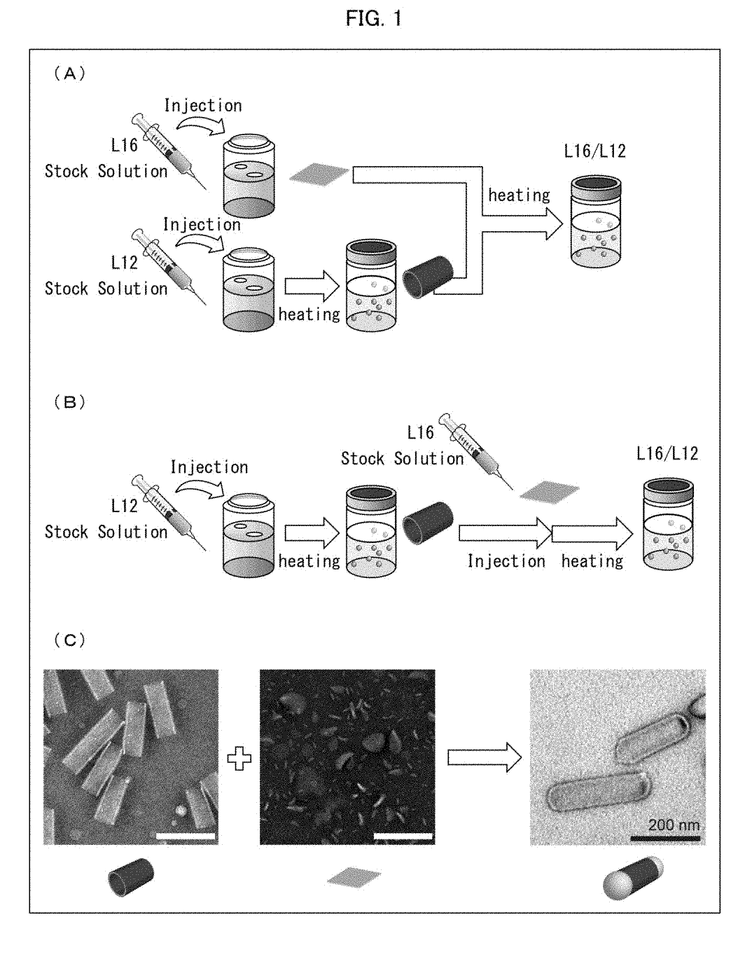

A capped nanostructure can be produced by, for example, a method illustrated in (A) of FIG. 1 or a method illustrated in (B) of FIG. 1. In FIG. 1, “L12” of Examples (described later) is used as first amphiphilic peptide chain, and “L16” of Examples (described later) is used as second amphiphilic peptide chain; however, this does not imply any limitation on the present embodiment.

In (A) of FIG. 1, the first amphiphilic peptide chain (L12) is dispersed in an aqueous medium and heated and thereby allowed to form a tube shape portion. Separately, the second amphiphilic peptide chain (L16) is dispersed in an aqueous medium and thereby allowed to form a sheet-shape structure. Next, the aqueous medium containing the sheet-shape structure and the aqueous medium containing the tube shape portion are mixed together and heated. This allows edges of the sheet-shape structure to associate with an end of the tube shape portion to form a cap portion.

In (B) of FIG. 1, the first amphiphilic peptide chain (L12) is first dispersed in an aqueous medium and heated, and thereby allowed to form a tube shape portion. Next, the second amphiphilic peptide chain is directly (that is, without allowing the second amphiphilic peptide chain to form a sheet-shape structure) added (for example, injected) to the aqueous medium containing the tube shape portion. The second amphiphilic peptide chain forms a sheet-shape structure in this aqueous medium containing the tube shape portion. Heating is carried out after the second amphiphilic peptide chain is added. This allows edges of the sheet-shape structure to associate with an end of the tube shape portion to form a cap portion.

In a case of producing nanostructures in the shape of capsules, it is preferable to employ Method (A) of FIG. 1 in order to obtain more uniformly sized cap portions, whereas it is preferable to employ Method (B) of FIG. 1 in order to achieve a higher yield of capped nanostructures.

The dispersion of the second amphiphilic peptide chain into an aqueous medium is carried out preferably at 4 to 25° C. It is also preferable that stirring is carried out in order to achieve a uniform dispersion and obtain a uniform sheet-shape structure.

The heating temperature is not particularly limited and is, for example, preferably 50 to 90° C. The heating time is not particularly limited and is, for example, 1 to 24 hours.

Second amphiphilic peptide chain suitable for preparation of a cap portion is, for example, the compound represented by the foregoing Formula (I) where, in one example, m is preferably 15 to 40 and n is preferably 7 to 10.

By appropriately selecting the kind of the first amphiphilic peptide chain and the kind of the second amphiphilic peptide chain, it is possible to obtain a peptide nanostructure that has high agent retaining ability. In one example, in a case where the first amphiphilic peptide chain is a compound represented by Formula (I) where m is 30 and n is 6 and the second amphiphilic peptide chain is a compound represented by Formula (I) where m is 30 and n is 8, a peptide nanostructure having high agent retaining ability can be obtained, as will be described later in Examples.

Although the above description dealt with a hollow body having a tube shape portion, the shape of the hollow body is not limited to such. The shape of the hollow body may be the shape of a rugby ball or the like.

Furthermore, although the above description dealt with an example case in which the amphiphilic molecule is an amphiphilic peptide chain, other cases may be employed in which the amphiphilic molecule is some other kind of molecule (lipid or the like).

(Encapsulation of Agent)

A nanostructure of the present embodiment encapsulates an agent therein. In this specification, the phrase “encapsulate an agent therein” means that an agent is not covalently bonded to a hollow body and that the agent is present in an inner space defined by the hollow body. Typically, an agent, which is dissolved or suspended in a liquid, is encapsulated. The liquid can be a hydrophilic liquid, and can be the foregoing aqueous medium.

A method of allowing an agent to be encapsulated in a hollow body is not particularly limited. A hollow body may be formed first and then an agent may be introduced into the inner space defined by the hollow body. In a case of an unclosed structure, the following method may be employed, for example: a hollow body is formed first; and then the hollow body is placed into a liquid (solution or suspension) containing an agent to allow the hollow body to encapsulate the agent therein. In one preferred example, a hollow body is allowed to form in a liquid (solution or suspension) containing an agent. For example, in a case where a hollow body is in the shape of a tube (unclosed structure), amphiphilic peptide chains are dispersed in an aqueous medium containing an agent and then heated to prepare a tube shape portion (hollow body). For example, in a case where a hollow body is in the shape of a capsule (closed structure), the first amphiphilic peptide chain is dispersed in an aqueous medium containing an agent and then heated to prepare a tube shape portion, and, to this dispersion, a sheet-shape structure of the second amphiphilic peptide chain ((A) of FIG. 1) or the second amphiphilic peptide chain ((B) of FIG. 1) is added and allowed to form cap portions, thereby obtaining a capsule shape (hollow body). In (A) of FIG. 1, the aqueous medium for use in formation of the sheet-shape structure of the second amphiphilic peptide chain may also contain the agent. That is, in one example, a method of producing a nanostructure of the present embodiment includes a step of preparing a tube shape portion by: dispersing amphiphilic peptide chains into an aqueous medium containing an agent to obtain a dispersion; and then heating the dispersion. This method makes it possible to allow an agent to be efficiently and easily encapsulated. The former is suitable for heat-labile agents (prone to alteration etc. by heat), whereas the latter is suitable for heat-resistant agents.

The size of the agent is not particularly limited, provided that the size of the agent is smaller than the inner diameter of the hollow body. The size of the agent is preferably equal to or less than 80 nm, more preferably equal to or less than 70 nm. In one example, the molecular weight of the agent is equal to or less than 50000, preferably equal to or less than 35000, more preferably equal to or less than 10000.

In one example, the agent is hydrophilic. The term “hydrophilic agent” also includes hydrophobic agents whose surface has been treated to be hydrophilic. None of the conventional nanostructures, which are intended for cellular uptake, have encapsulated a hydrophilic agent. As will be described later in Examples, the inventors for the first time succeeded in preparing a nanostructure that encapsulates a hydrophilic agent therein.

Examples of the agent include effective ingredients of pharmaceuticals and food (in particular, functional food), effective ingredients in the field of cosmetics, molecular probes for imaging systems, and various research reagents. The agent can be an organic compound, inorganic compound, biomolecule such as protein or nucleic acid, or the like. A single kind of agent may be encapsulated in the nanostructure or two or more kinds of agent may be encapsulated in the nanostructure. It should be noted that the nanostructure of the present embodiment excludes those in which the encapsulated agent is in the form of saline solution (e.g., physiological saline) (that is, the agent itself is saline solution or the agent is sodium chloride dissolved in water in liquid form) and the nanostructure does not contain components other than the agent (saline solution). The nanostructure of the present embodiment enables efficient cellular uptake of a drug which alone had not been taken up by cells or a drug which alone had been difficult to be taken up by cells.

The peptide nanostructure of the present embodiment contains peptide within its structure and thus is biodegradable. Therefore, the peptide nanostructure is decomposed in a living organism (for example, cell) and the agent is released in the living organism (for example, cell). In one example, the release of the agent can continue, for example, for 1 day or more, 2 days or more, or 4 days or more. The biodegradation can take place due to, for example, protease such as proteinase or peptidase.

As will be described later in Examples, the nanostructure of the present embodiment can be taken up through endocytosis mediated by clathrin in an energy dependent manner (note, however, that this does not imply any limitation). Clathrin is a protein that many biological species have, and therefore the peptide nanostructure of the present embodiment can be used in many biological species.

According to the peptide nanostructure of the present embodiment, by changing the kind of amphiphilic peptide chain, it is possible to adjust the size, shape, tissue selectivity, and rate of decomposition in vivo of the peptide nanostructure, release characteristics (controlled release property or the like) of the encapsulated agent, and/or the like.

(Other Applications)

The present embodiment also provides a pharmaceutical composition containing a nanostructure. The pharmaceutical composition contains a medicament as the agent. Any medicament can be used without particular limitation according to a target disease. Specific examples of the medicament include anticancer agents, antibacterial agents, antiviral agents, anti-inflammatory agents, immunosuppressive agents, steroids, hormones, and anti-angiogenic agents.

The route of administration of the pharmaceutical composition is not particularly limited. The pharmaceutical composition may be administered systemically by oral administration, intravascular administration such as intravenous administration or intraarterial administration, enteral administration, or the like, or may be administered topically by transdermal administration, sublingual administration, or the like. In one example, the pharmaceutical composition is preferably administered by intravenous injection. The dose of the pharmaceutical composition administered to a patient may be selected appropriately according to the kind of the encapsulated medicament, age, gender, body weight, and condition of the patient, route of administration, frequency of administration, administration period, and the like. A target organism that receives the administration is not particularly limited as well. Examples include plants and animals. Animals such as fishes, amphibians, reptiles, birds, and mammals are preferred, and mammals are more preferred. Mammals are not limited to a particular kind, and examples include: laboratory animals such as mice, rats, rabbits, guinea pigs, and non-human primates; pets such as dogs and cats; domestic animals such as cattle, horses, and pigs; and humans.

The dosage form of the pharmaceutical composition is not particularly limited, and can be a solution obtained by dispersing a nanostructure in a hydrophilic liquid. Examples of the hydrophilic liquid include water, alcohols, and buffer solutions. The pharmaceutical composition may further contain a preservative, a stabilizer, a buffer agent, an osmotic adjuster, a colorant, a flavoring agent, a sweetener, an antioxidant, a viscosity modifier, and/or the like, in addition to the nanostructure.

The nanostructure of the present embodiment encapsulates an agent therein, is easily taken up by cells, and can release the agent (for example, release in a controlled manner) in the cells. As such, the pharmaceutical composition of the present embodiment is capable of efficiently delivering a medicament into cells as compared to cases in which the pharmaceutical composition is administered alone. Thus, lower doses can be enough to provide the effect of the medicament for long time.

(Specific Examples of Configurations in Accordance with the Present Invention)

One aspect of the present invention includes the following.

(1) A nanostructure which is a hollow body constituted by a wall formed from an assembly of amphiphilic molecules containing a hydrophilic block and a hydrophobic block, the hollow body having an aspect ratio greater than 1.0,

the nanostructure encapsulating an agent therein.

(2) The nanostructure according to (1), wherein the aspect ratio is in the range of from 1.2 to 30.0.
(3) The nanostructure according to (1) or (2), wherein the hollow body includes a tube shape portion.
(4) The nanostructure according to any of (1) to (3), wherein the hollow body has a closed structure.
(5) The nanostructure according to any of (1) to (4), wherein the amphiphilic molecules are amphiphilic peptide chains containing a hydrophilic peptide block and a hydrophobic peptide block.
(6) The nanostructure according to (5), wherein the hydrophobic block has a helix structure.
(7) The nanostructure according to (5) or (9), wherein the hydrophobic peptide block contains leucine-aminoisobutyric acid as repeating unit.
(8) The nanostructure according to any of (5) to (7), wherein the hydrophilic peptide block contains sarcosine as repeating unit.
(9) The nanostructure according to any of (1) to (8), wherein the agent is a hydrophilic agent.
(10) A pharmaceutical composition comprising the nanostructure recited in any of (1) to (9).
(11) A method of producing the nanostructure recited in any of (1) to (9), the method including a step of preparing a tube shape portion by: dispersing the amphiphilic molecules containing a hydrophilic block and a hydrophobic block into an aqueous medium containing the agent to obtain a dispersion; and then heating the dispersion.

The following will provide Examples to more specifically describe embodiments of the present invention. As a matter of course, the present invention is not limited to Examples provided below, but details of the present invention can be realized in various manners. Further, the present invention is not limited to the embodiments described above, and it may be varied in various ways within the scope of the appended claims. Thus, an embodiment based on a combination of technical means disclosed in different embodiments is encompassed in the technical scope of the present invention. Furthermore, all of the publications and patents cited in the present specification are incorporated herein by reference in their entirety.

### CROSS-REFERENCE OF RELATED APPLICATIONS

This application claims priority on Japanese Patent Application, Tokugan, No. 2017-096075 filed in Japan on May 12, 2017, the entire contents of which are hereby incorporated by reference.

### EXAMPLES

### Example 1: Production of Nanocapsule

Amphiphilic peptide Sar₃₀-(L-Leu-Aib)₆ and Amphiphilic peptide Sar₃₀-(L-Leu-Aib)₈, in each of which the hydrophilic block is constituted by polysarcosine and the hydrophobic block is constituted by an α-helix of poly(L-leucine-aminoisobutyric acid), were prepared in accordance with a previous report (Document: M Ueda et al., Chem. Commun. 47, 3204-3206 (2011)). The amphiphilic peptides Sar₃₀-(L-Leu-Aib)₆ and Sar₃₀-(L-Leu-Aib)₈ are hereinafter referred to as “L12” and “L16”, respectively.

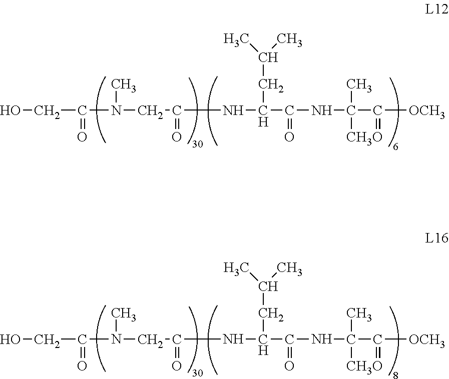

Next, 40 mg of L12 or L16 was dissolved in 800 μL of ethanol. In this way, stock solutions of L12 and L16, respectively, were obtained.

10 μL of the L12 stock solution was added (injected) to 990 μL of physiological saline and dispersed with stirring at 25° C. for 30 minutes. The resultant dispersion was heated at 80° C. or 90° C. for 1 hour to 7 hours and cooled to room temperature, with the result that nanotubes formed. The nanotubes had an aspect ratio which changed depending on heating conditions. The aspect ratio was 1.5 (about 80 nm in diameter and about 120 nm in length) in the case of heating at 80° C. for 1 hour, 2.4 (about 80 nm in diameter and about 200 nm in length) in the case of heating at 80° C. for 3 hours, 3.8 (about 80 nm in diameter and about 310 nm in length) in the case of heating at 90° C. for 1 hour, and 7.0 (about 80 nm in diameter and about 560 nm in length) in the case of heating at 90° C. for 3 hours.

10 μL of the L16 stock solution was added (injected) to 990 μL of physiological saline and dispersed with stirring at 25° C. for 30 minutes. L16 formed nanosheets in a case where heating was not carried out. On the other hand, in a case where a heat treatment (at 90° C. for 1 hour) was carried out, L16 turned into nanospheres 80 nm in diameter.

The dispersion containing the L16 nanosheet was added to the dispersion containing the L12 nanotube ((A) of FIG. 1) or the L16 stock solution was added (injected) to the dispersion containing the L12 nanotube ((B) of FIG. 1), and gently dispersed for 30 seconds. The resultant solution was heated at 80° C. for 3 hours. The ratio by weight of L12 to L16 was 1:2.

TEM images were taken using a JEOL JEM-1230 at an accelerating voltage of 80 kV. A drop (2 μL) of the dispersion was mounted on a carbon-coated Cu grid and stained negatively with 2% samarium acetate, followed by suction of the excess fluid with filter paper. Frozen-Hydrated/Cryogenic-TEM (Cryo-TEM) observation was performed. The dispersions in buffer solutions were frozen quickly in liquid ethane, which was cooled with liquid nitrogen. The samples were examined at 100 kV accelerating voltage at the liquid nitrogen temperature.

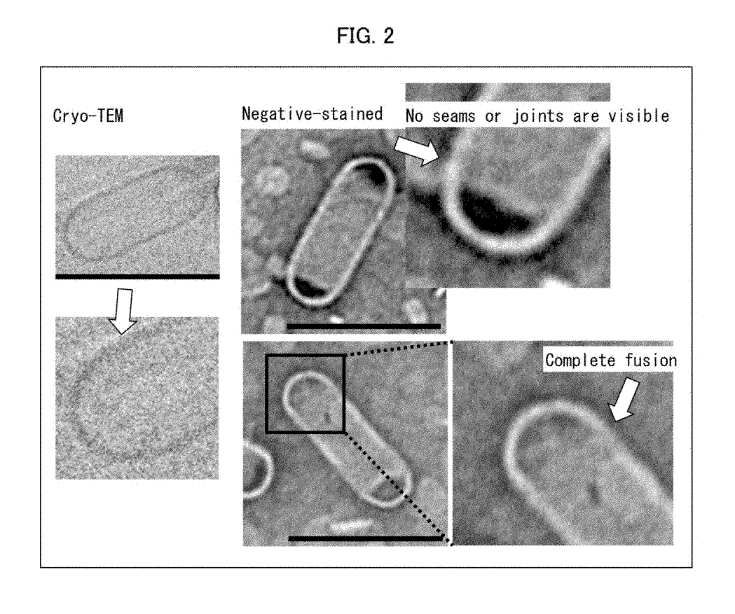

As illustrated in FIG. 2, openings of the L12 nanotube were sealed with the L16 nanosheet, which resulted in nanocapsule formation. Both Method (A) of FIG. 1 and Method (B) of FIG. 1 resulted in the formation of good-quality nanocapsule with good yield. Method (A) of FIG. 1 provided a smaller percentage of dumbbell shape than Method (B) of FIG. 1, and cap portions obtained by Method (A) of FIG. 1 were more uniform in size than Method (B) of FIG. 1. On the other hand, Method (B) of FIG. 1 gave capped peptide nanostructure with a better yield than Method (A) of FIG. 1.

### Example 2: Evaluation of Drug Retaining Ability

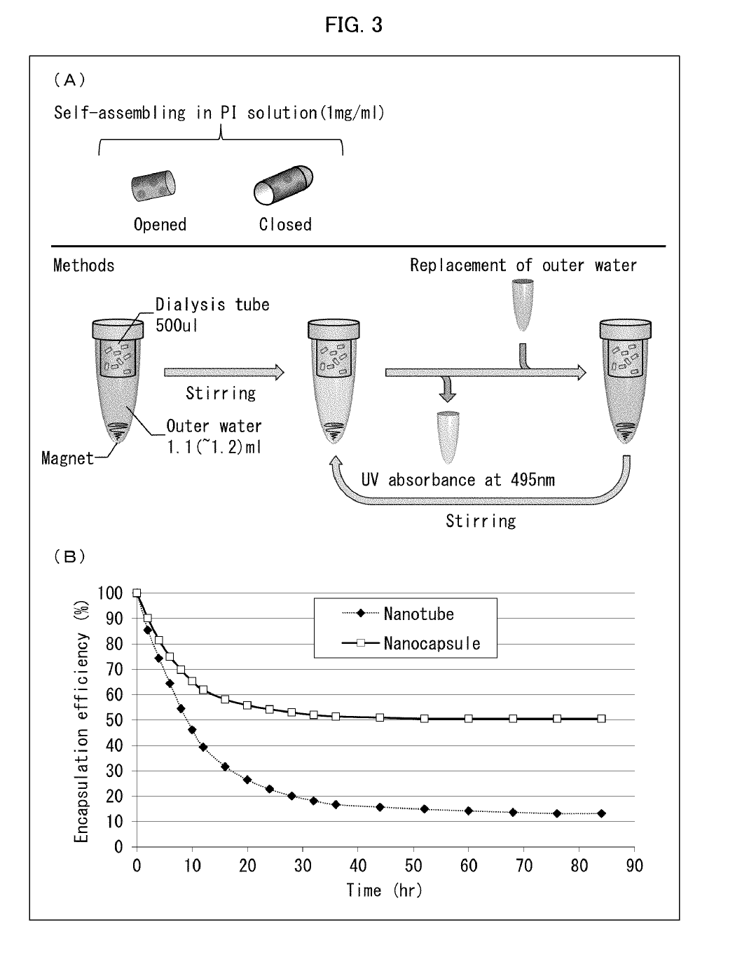

Self-assembly was allowed to take place in a propidium iodide (PI) solution (1 mg/mL) instead of physiological saline, thereby preparing PI-encapsulating nanotube and PI-encapsulating nanocapsule. L12 was heated at 80° C. for 3 hours, and the addition of L16 was carried out by Method (B) of FIG. 1. Next, in a dialysis tube (MWCO 10K, Slide-A-Lyzer MINI dialysis unit, 25 mL), the PI-encapsulating nanotube and PI-encapsulating nanocapsule were purified ((A) of FIG. 3). The amount of escaped PI through a dialysis membrane was calculated by UV absorbance at 495 nm over dialysis time.

The results are shown in (B) of FIG. 3. For the first several tens of hours, the PI dissolved in outer solution was released and therefore the encapsulation efficiency rapidly decreased in both cases. In regard to the uncapped nanostructure, the encapsulation efficiency continued to gradually decrease even after the rapid decrease, and it was found that almost entire PI escaped from the nanostructure. On the other hand, in regard to the capped nanostructure, the escape of PI was not observed after the escape of PI dissolved in the outer solution, and it was found that the encapsulated PI was kept in the nanostructure.

### Reference Example 1: Relationship Between Aspect Ratio of Nanostructure and Cellular Uptake

Cellular uptake assay of nanostructures was carried out using fluorescent nanostructures which contained 1% fluorescent peptides (FITC conjugated with N-terminus of hydrophobic helical block). The nanostructures used here were nanotube having an aspect ratio of 1.5 (about 80 nm in diameter and about 120±20 nm in length), nanotube having an aspect ratio of 2.4 (about 80 nm in diameter and about 200±20 nm in length), nanotube having an aspect ratio of 3.8 (about 80 nm in diameter and about 310±50 nm in length), and nanotube having an aspect ratio of 7.0 (about 80 nm in diameter and about 560±160 nm in length), each of which was prepared using Sar₃₀-(L-Leu-Aib)₆. As nanostructure for comparison, nanosphere about 100 nm in diameter was used. The nanosphere was prepared in the following manner. L16 was dissolved in ethanol to prepare a stock solution (0.05 mg/μL). 10 μL of this stock solution was injected into physiological saline (1 mL) of 4 to 25° C. and gently stirred for 30 minutes. Then, the resultant solution was treated with heat at 90° C. for 1 hour to obtain nanosphere.

HeLa cell was seeded in 48-well plates at a density of 8×10³ cells per well (160 μL) with 1% FBS in DMEM and incubated for 12 hours at 37° C. in 5% CO₂ atmosphere. 40 μL of a fluorescent nanostructure solution (0.5 mg/mL in PBS) was added to each well and incubated for 1 hour at 4° C. or 37° C. in 5% CO₂ atmosphere. Cells were washed with PBS twice and incubated with 4% paraformaldehyde in PBS for 10 minutes at room temperature, protecting from light. Cells were washed with 3% FBS in PBS 3 times and imaged under a fluorescent microscope (Axio Observer. Z1, ZEISS). Quantitative analysis of the fluorescent intensity was performed by image analysis software Image J.

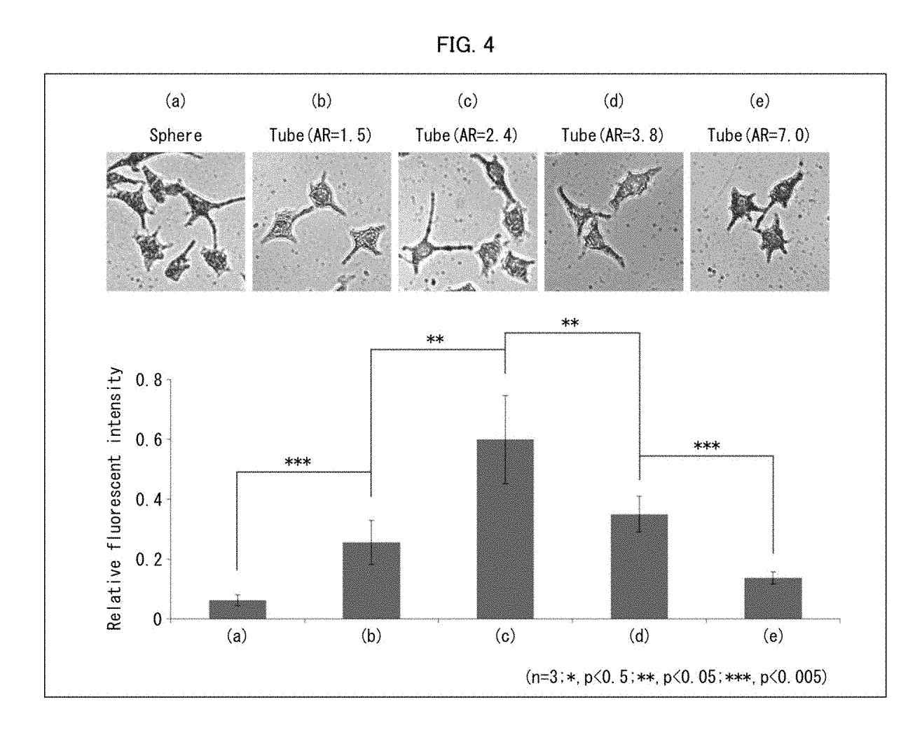

The functions of cells are suppressed at 4° C., and therefore each fluorescent nanostructure was almost not taken up. On the other hand, at 37° C., cellular uptake of nanotube was greater than cellular uptake of nanosphere (FIG. 4). The amount of cellular uptake was especially large when the aspect ratio was 2.4 to 3.8.

### Reference Example 2: Study on Mechanism of Cellular Uptake

Cells were incubated with various chemicals to inhibit cellular uptake pathways. HeLa cell was seeded in 48-well plates at a density of 1×10⁴ cells per well (160 μL) with 1% FBS in DMEM and incubated for 12 hours at 37° C. in 5% CO₂ atmosphere. Cells were incubated with chlorpromazine (10 μg/mL), Filipin III (1 μg/mL), or amiloride (50 nM) for 30 minutes at 37° C. in 5% CO₂ atmosphere or at 4° C. Fluorescent nanostructures prepared in the foregoing manner were added to the medium and incubated further 2 hours. Cells were washed with PBS twice and fixed by 4% paraformaldehyde (in PBS) at room temperature for 10 minutes, protecting from light. Cells were washed with 3% PBS twice and imaged under the fluorescent microscope.

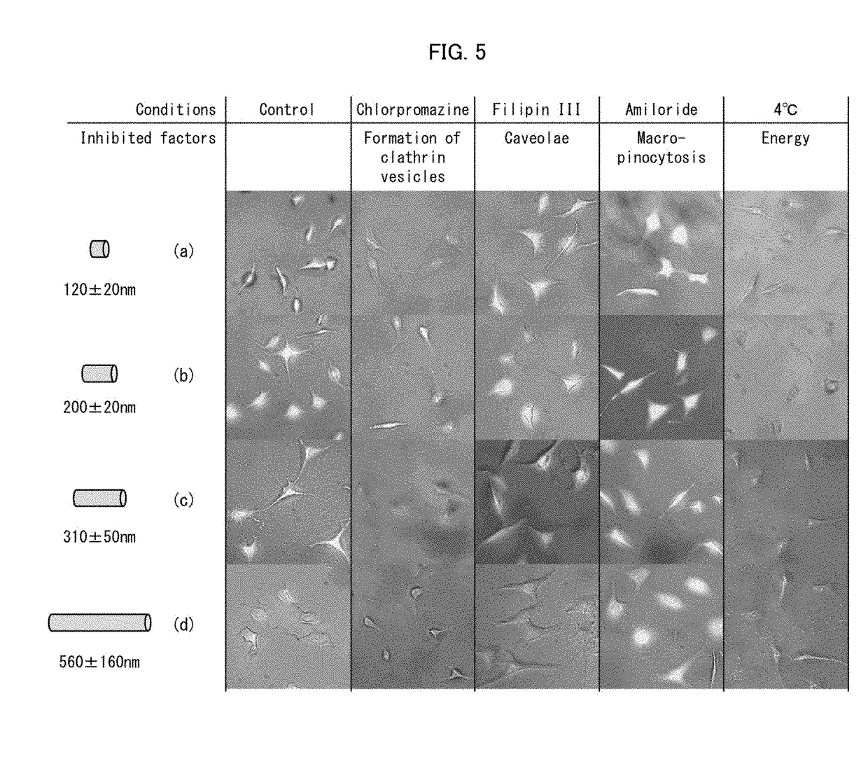

The results are shown in FIG. 5. When chlorpromazine was used, no cellular uptake was observed at any aspect ratio. Chlorpromazine inhibits the formation of clathrin vesicles that form on the inner surface of the cell membrane during endocytosis by cells. Also in the case of 4° C., there was almost no cellular uptake. These results reveled that these nanostructures were taken up through endocytosis mediated by clathrin in an energy dependent manner.

### Reference Example 3: Confirmation of Location of Nanostructure in Cell

With a confocal laser microscope, the location of FITC-labelled nanostructure in the foregoing HeLa cell after incubation of 1 hour at 37° C. was confirmed.

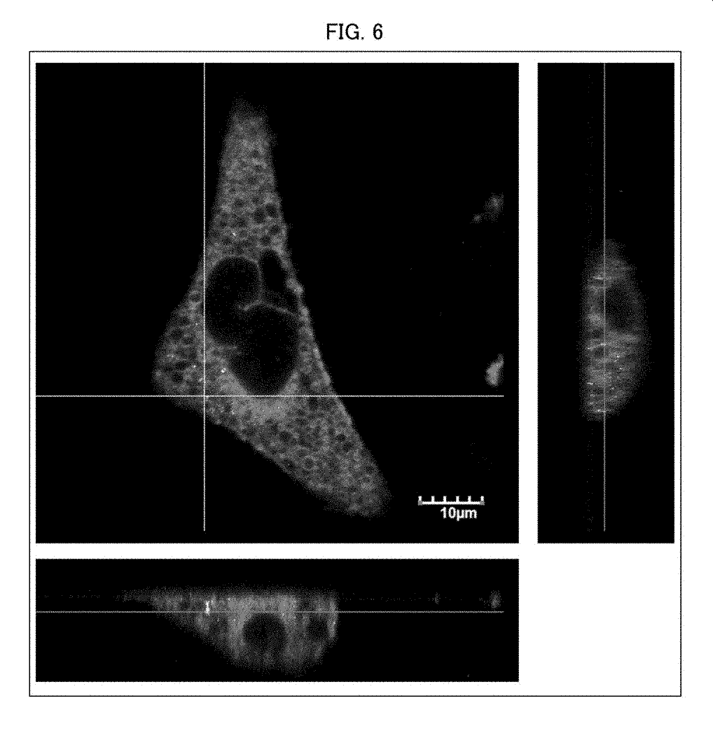

The result is shown in FIG. 6. It was confirmed that the nanotube was present in a cell.

### Example 3: Test 1 on Delivery of Agent by Nanocapsule

PI-encapsulating nanocapsule was prepared in the same manner as described in Example 2. The PI-encapsulating nanocapsule dispersion was incubated with HeLa cell for 1 hour and washed with a buffer solution several times to remove the nanocapsule and free PI outside completely. The location of PI was evaluated by fluorescent microscope observation. PI alone incubated with HeLa cell for 1 hour was used as a negative control.

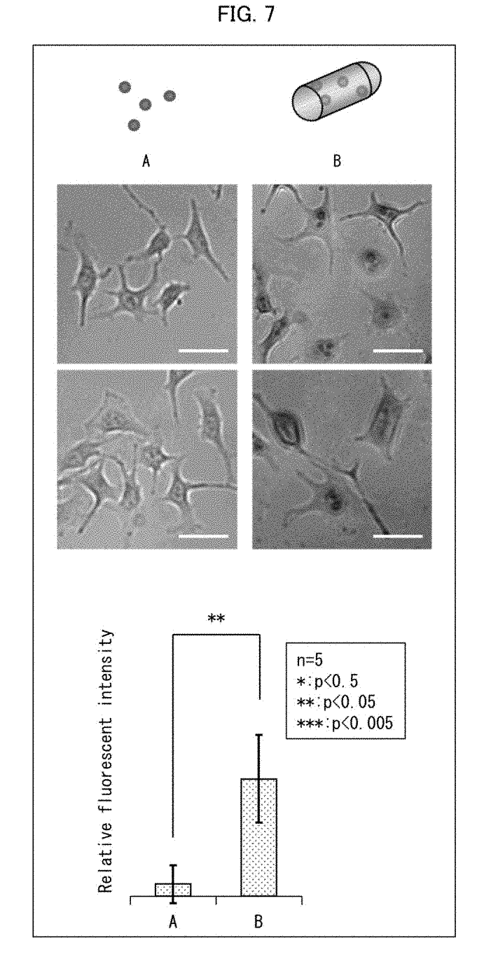

The results are shown in FIG. 7. It was confirmed that the PI alone was not taken up by cells, whereas the PI-encapsulating nanocapsule was taken up by cells.

### Example 4: Test 2 on Delivery of Agent by Nanocapsule

Nanocapsule and nanosphere were each labelled with indocyanine green (ICG) by attaching ICG-EG-sulfo8-NHS (Dojindo, Japan) to nanocapsule and nanosphere. Specifically, the ICG-labeled nanocapsule was prepared in the following manner: L12 with free N-terminus, which was not protected by ketole group, was mixed in L12 at a mixing ratio of 1 mol %; the same process as described in Example 1 was carried out to prepare nanocapsule; and then ICG was attached to the terminus. L12 was heated at 80° C. for 3 hours, and the addition of L16 was carried out by Method (B) of FIG. 1. The ICG-labeled nanosphere was prepared in the following manner: L16 with free N-terminus, which was not protected by ketole group, was mixed in L16 at a mixing ratio of 1 mol %; the same process as described in Reference Example 1 was carried out to prepare nanosphere; and then ICG was attached to the terminus.

Tumor was transplanted into 6-week old mice using EL4 cell (1×10⁶ cells/body). After 4 days feeding, transplanted tumors were identified and ICG-labelled nanostructures were injected through tail vein. The cancer accumulation of ICG-labelled nanocapsule was evaluated by infrared imaging of IVIS imaging system (PerkinElmer, USA) (n=2). Hair was cut and removed before infrared imaging.

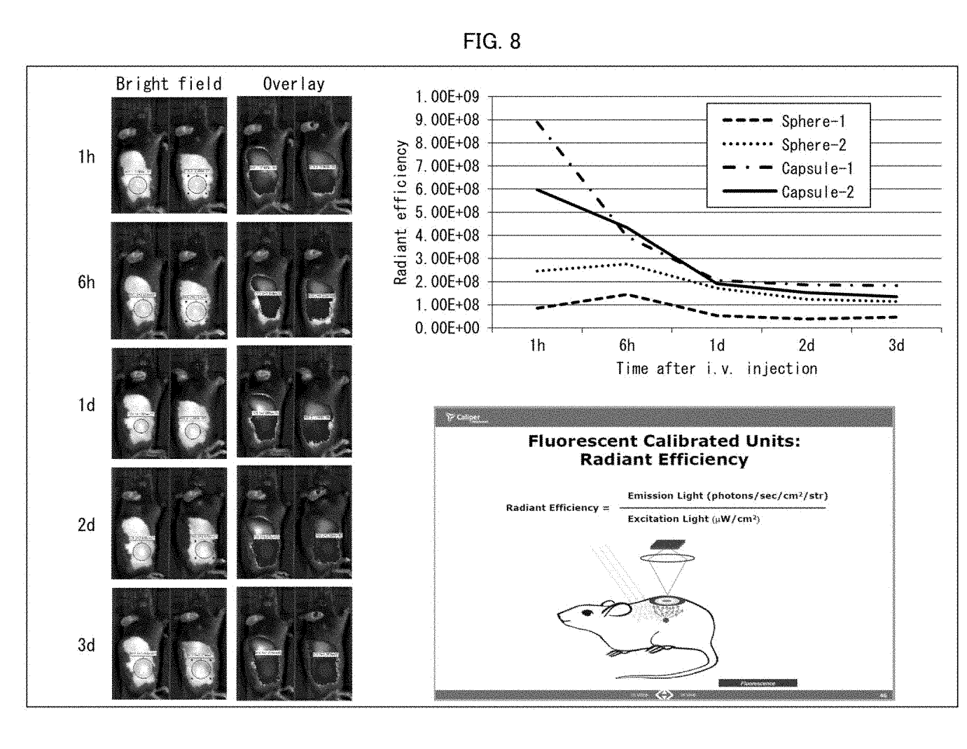

The results are shown in FIG. 8. The results showed that nanocapsule accumulates in a tumor site more rapidly than nanosphere. The results also revealed that nanocapsule accumulates in a tumor site in a greater amount than nanosphere.

### Example 5: Test 3 on Delivery of Agent by Nanocapsule

Self-assembly was allowed to take place in the anticancer agent cisplatin solution (1 mg/mL in PBS) instead of physiological saline, thereby preparing cisplatin-encapsulating nanocapsule and cisplatin-encapsulating nanosphere. The conditions under which the nanocapsule was produced were the same as those of Example 2. The conditions under which the nanosphere was produced were the same as those of Reference Example 1. Tumor was transplanted into 6-week old mice using EL4 cell (1×10⁶ cells/body). After 4 days feeding, transplanted tumors were identified and cisplatin-encapsulating nanostructures were injected through tail vein. Tumor volume and body weight changes of mice were recorded every other day. Those which received an injection of a buffer alone were used as a control. Those which received an injection of cisplatin alone were used for comparison.

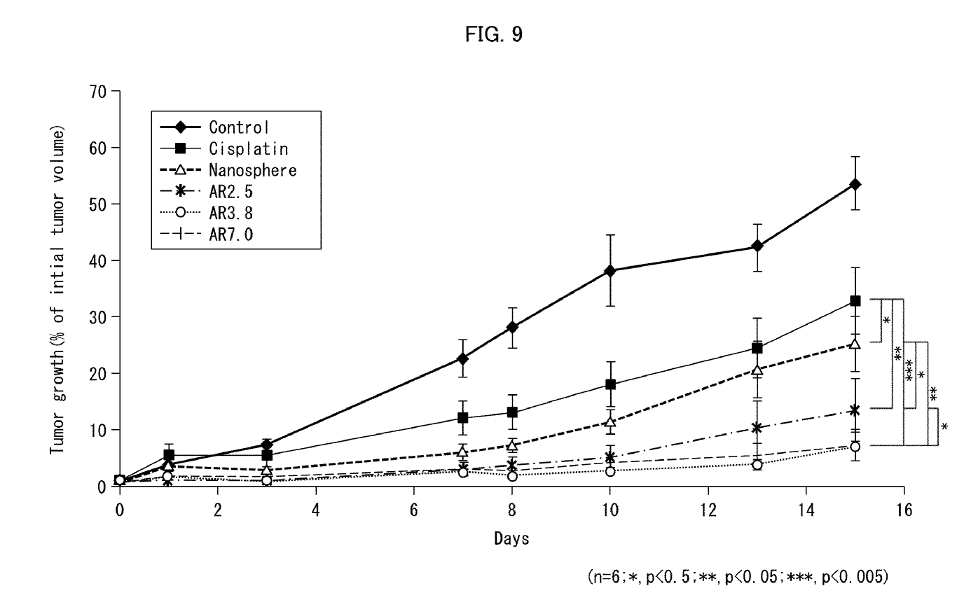

The results are shown in FIG. 9. The nanocapsule showed the long-lasting effect of inhibiting tumor growth as compared to the case of cisplatin alone and the case of nanosphere.

### Example 6: Test 1 on Decomposition of Nanostructure

The L12 nanotube was prepared in the same manner as described in Example 1 under the heating conditions of 80° C. and 3 hours. The nanotube was incubated with proteinase K (30 U/mL) in 50 mM of Tris-HCl containing 5 mM of CaCl₂. TEM images were taken in the same manner as described in Example 1.

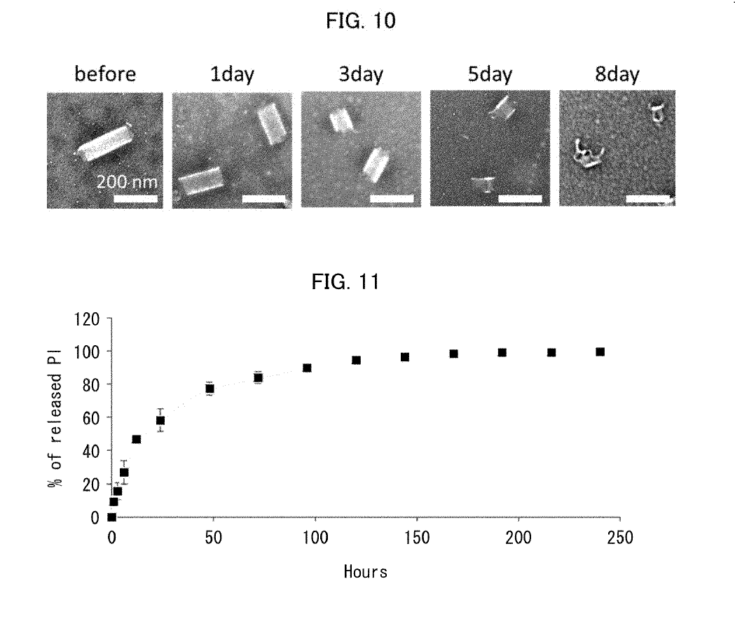

The results are shown in FIG. 10. The results showed that the nanotube undergoes biodegradation as time passes.

### Example 7: Test 2 on Decomposition of Nanostructure

The PI-encapsulating nanocapsule prepared in Example 2 was incubated with proteinase K (30 U/mL) in 50 mM of Tris-HCl containing 5 mM of CaCl₂ in a dialysis tube (MWCO 10K, Slide-A-Lyzer MINI dialysis unit, 25 mL), and dialyzed in 50 mM of Tris-HCl containing 5 mM of CaCl₂. The amount of escaped PI through a dialysis membrane was calculated by UV absorbance at 495 nm over incubation time.

The results are shown in FIG. 11. FIG. 11 shows the relationship between time after addition of proteinase K and the amount of released PI. The results showed that the nanocapsule undergoes biodegradation and the encapsulated drug is released as time passes.

### INDUSTRIAL APPLICABILITY

A nanostructure of the present invention can be used widely in the fields of pharmaceuticals, food, cosmetics, and the like as, for example, a carrier to transport an agent into cells.

1. A nanostructure which is a hollow body constituted by a wall formed from an assembly of amphiphilic molecules containing a hydrophilic block and a hydrophobic block, the hollow body having an aspect ratio greater than 1.0,
the nanostructure encapsulating an agent therein.

2. The nanostructure according to claim 1, wherein the aspect ratio is in the range of from 1.2 to 30.0.

3. The nanostructure according to claim 1, wherein the hollow body includes a tube shape portion.

4. The nanostructure according to claim 1, wherein the hollow body has a closed structure.

5. The nanostructure according to claim 1, wherein the amphiphilic molecules are amphiphilic peptide chains containing a hydrophilic peptide block and a hydrophobic peptide block.

6. The nanostructure according to claim 5, wherein the hydrophobic block has a helix structure.

7. The nanostructure according to claim 5, wherein the hydrophobic peptide block contains leucine-aminoisobutyric acid as repeating unit.

8. The nanostructure according to claim 5, wherein the hydrophilic peptide block contains sarcosine as repeating unit.

9. The nanostructure according to claim 1, wherein the agent is a hydrophilic agent.

10. A pharmaceutical composition comprising the nanostructure recited in claim 1.

11. A method of producing the nanostructure recited in claim 1, the method comprising a step of preparing a tube shape portion by: dispersing the amphiphilic molecules containing a hydrophilic block and a hydrophobic block into an aqueous medium containing the agent to obtain a dispersion; and then heating the dispersion.
